(19) United States
(12) Patent Application Publication
(10) Pub. No.: US 2004/0147989 A1
(43) Pub. Date: Jul. 29, 2004

(54) Therapeutic body area-specific covering

(76) Inventors: Tim Terakita, Winter Park, FL (US); Lisa Randall, Winter Park, FL (US); James A. Bucci, Oviedo, FL (US); Kyra Bucci, Oviedo, FL (US)

Correspondence Address: BEUSSE BROWNLEE WOLTER MORA & MAIRE, P. A., 390 NORTH ORANGE AVENUE, SUITE 2500, ORLANDO, FL, 32801, US

(21) Appl. No.: 10/649,420

(22) Filed: Aug. 27, 2003

Related U.S. Application Data
(60) Provisional application No. 60/442,573, filed on Jan. 27, 2003.

(51) Int. Cl. A61F 7/00

(52) U.S. Cl. 607/108; 607/112; 607/114

(57) ABSTRACT

A therapeutic body covering specific to a certain area of a body, the body covering comprising a panel for covering a certain area of a body, a removable flap positioned over the panel, a therapeutic insert configured to conform to the area of the body, a securing panel to hold the panel to the certain area of the body, and wherein the panel and the securing panel are configured so that the certain area of the body is not moved when placing the therapeutic insert between and removing the therapeutic insert from the panel and the flap.

This application claims priority of Provisional Patent Application Serial No. 60/442,573, filed on Jan. 27, 2003.

### BACKGROUND

This invention relates to a body covering, and more specifically to a therapeutic body covering specific to a certain area of a body where the covering is used in applying cold and heat to a specific part of a body.

Therapeutic body coverings where either heat or cold needs to be applied are used for a variety of reasons, such as, but not limited to, injuries sustained, natural body deterioration, and recovery from a surgery. Individuals recovering from sports injuries and occupational injuries, or natural body deterioration, such as arthritis, where soreness or other external pain is realized, are sometimes directed by their medical care provider to apply either a heat of cold compress to the effected area. Similarly, depending on a type of surgery an individual undergoes, applying a heat or cold compress may also be a recommended recovery procedure.

One example of where a medical care provider will recommend applying cold compresses after a surgery is with respects to breast augmentation. Breast augmentation, such as breast enlargement or breast reduction, is a growing business for plastic surgeons. While the surgical procedure of breast augmentation has been advanced, the procedure for post-operative and recovery time care has not. Typically, after a plastic surgeon completes augmentation, the surgical team will place a surgical bra on the patient while the patient is still unconscious. Once conscious and leaving the hospital to continue recovery, the patient is typically directed to apply ice to the breast area, but not directly onto the skin at a certain interval, such as ten-minute intervals, for at least 48 hours following the surgery. This is done to help reduce the swelling and discomfort realized by the patient.

Currently, the recommended procedure for cooling the breast area is to use packages of frozen vegetables, or berries, that are roughly the size of peas. The packages of frozen produce are to be placed on the breast area over the surgical bra. Depending on the brand of frozen produce used, applying frozen produce can be somewhat messy as the produce begins to thaw. For example, with respect to peas, if the package that the peas are in is not properly sealed, the liquid from the peas could seep through the package and onto the patient or onto and through the surgical bra. Placing the frozen produce upon the breast area is also somewhat difficult, since a minimal amount of movement of the breast is preferred, and properly situating the frozen produce so that the package does not move or stays in a desired location is also somewhat difficult to do, depending upon the breast size.

Breast engorgement is a common occurrence that breast feeding mothers may encounter if the intervals between breast feeding are not at regular intervals where the interval between feedings is extended. One of the recommended treatments for breast engorgement is to apply a warm compress to the engorged breast. Such techniques for applying a warm compress include applying a warm washcloth or warm water bottle. However such techniques are not conducive to a mother who has to travel or who seeks relief while not in a private location to apply such treatments.

### SUMMARY OF THE INVENTION

The therapeutic body covering provides for a covering that is specially designed to allow its wearer mobility while still providing for a heating and/or cooling effect wherein applying the heating and/or cooling effect will result in minimum movement of the area of the body covered when the heating and/or cooling effect is being applied. Towards this end, a therapeutic body covering specific to a certain area of a body is disclosed. Such body areas include, but are not limited to a breast area, elbow area, knee area, shoulder area, and ankle area. The body covering comprises a panel for covering a certain area of a body and a removable flap positioned over the panel. A therapeutic insert configured to conform to the area of the body is also provided for, as well as a securing panel to hold the panel to the certain area of the body. The panel and the securing panel are configured so that the certain area of the body that is covered is not moved when placing the therapeutic insert between and removing the therapeutic insert from the panel and the flap.

With respect to the mammary glands, a therapeutic bra is disclosed. The therapeutic bra comprises a first front panel for covering a first breast and a second front panel for covering a second breast. A closure device is provided for holding the first panel and the second panel together. A back panel is also included with a first end connected to the front panel and a second end connected to the second panel. A removable first flap is positioned over the first front panel and a removable second flap positioned over the second front panel. A therapeutic insert is provided wherein the therapeutic insert is configured to conform to a breast size. The first front panel, the second front panel, and the back panel are configured so that the breasts are not moved when placing the therapeutic insert between and removing the therapeutic insert either the first front panel with the first flap or the second front panel with the second flap.

A therapeutic bra for reducing movement of a breast when applying a cooling compress and/or a warming compress to the breast is also disclosed. The bra has a first bra cup covering a first breast and a second bra cup covering a second breast. A first bra cup flap removable from an outer edge of the first bra cup and a second bra cup flap removable from an outer edge of the second bra cup are also included. The bra also has a back strap having a first end connected to the first bra cup and a second end connected to the second bra cup. Either a cooling compress and/or a warming compress formed to a shape of either the first breast or the second breast is also provided wherein the cooling compress and/or the warming compress fits between at either the first bra cup flap connected to the first bra cup or the second bra cup flap connected to the second bra cup.

Also further disclosed herein is an improvement to a bra having a first bra cup and a second bra cup. The improvement comprises a first movable flap covering a first bra cup, a second movable flap covering a second bra cup, and a therapeutic inserts that fits between each first flap in combination with the first bra cup and the second flap in combination with the second bra cup. Each flap can be opened for insertion and removal of the therapeutics where a breast is not moved while removal or insertion of the therapeutic insert is taking place.

### BRIEF DESCRIPTION OF THE DRAWINGS

The features and advantages of the present invention will become apparent for the following detailed description of the invention when read with the accompanying drawings in which:

FIG. 1 is an exemplary embodiment of a therapeutic bra illustrating a therapeutic insert and removable flap;

FIG. 2 is an exemplary embodiment of a therapeutic bra used for breast-feeding;

FIG. 3 is another exemplary embodiment of a therapeutic bra used for breast-feeding;

FIG. 4 is an exemplary embodiment of a therapeutic bra wherein the strap is removable from the bra;

FIG. 5 is an elevational view of an exemplary embodiment of a gel pack that fits within a therapeutic bra;

FIG. 6 is an exemplary embodiment of a therapeutic body covering specific to a shoulder;

FIG. 7 is an exemplary embodiment of a therapeutic body covering specific to a knee; and

FIG. 8 is an exemplary embodiment of a therapeutic body covering specific to an elbow.

### DETAILED DESCRIPTION OF THE INVENTION

Before describing in detail various aspects of the present invention, it should be observed that the present invention broadly comprises a novel combination of components configured to apply a cold compress and/or a hot (or warm) compress to a body part. Though the present disclosure is explained in detail for use with the breast area, or mammary glands, where a user is undergoing discomfort with the breast area of the body, the present invention may be used for numerous other uses. For example, the structural details or operational interrelationships are also applicable to other parts of a body, such as but not limited to, a leg area such as a knee, an arm area such as an elbow, a foot area such as an ankle, and/or a torso area such as a shoulder. Accordingly, the components disclosed in the drawings show only those specific details that are pertinent to the present invention so as not to obscure the disclosure, structural details or operational interrelationships that will be readily apparent to those skilled in the art having the benefit of the description herein. Furthermore, though the invention is disclosed for use with breast augmentation or when other discomfort is realized around the breast area, such as engorgement during breast-feeding, the present invention has other applications as well, such as a prosthetics holder for those experiencing breast removal, and should not be limited to those disclosed in the present invention. Similarly the present invention is not limited to use on humans since the present invention may be applied to animals as well. With reference to the drawings, exemplary embodiments of the invention will now be described.

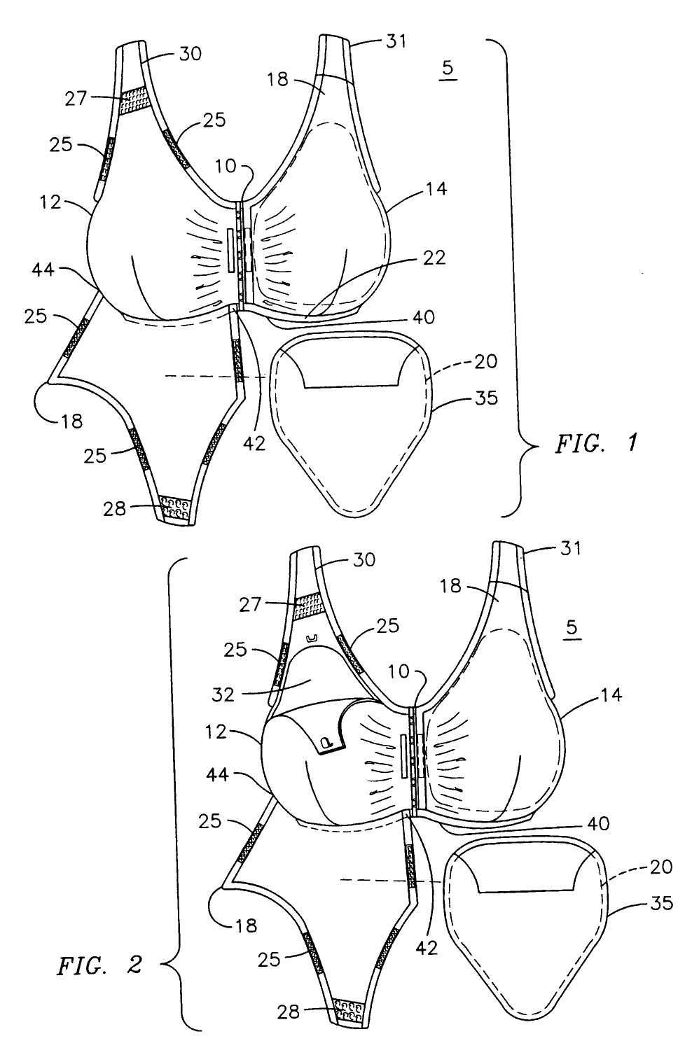

FIG. 1 is an exemplary embodiment of a therapeutic bra illustrating a therapeutic insert and removable flap. As illustrated in FIG. 1, the therapeutic bra 5 is a front-opening/closure bra where the opening/closure 6 is located between a right breast covering 12 and a left breast covering 14, or left and right bra cups. The bra 5 is a full support bra. The shape and size of the bra 5 may vary to accommodate various bra cup sizes.

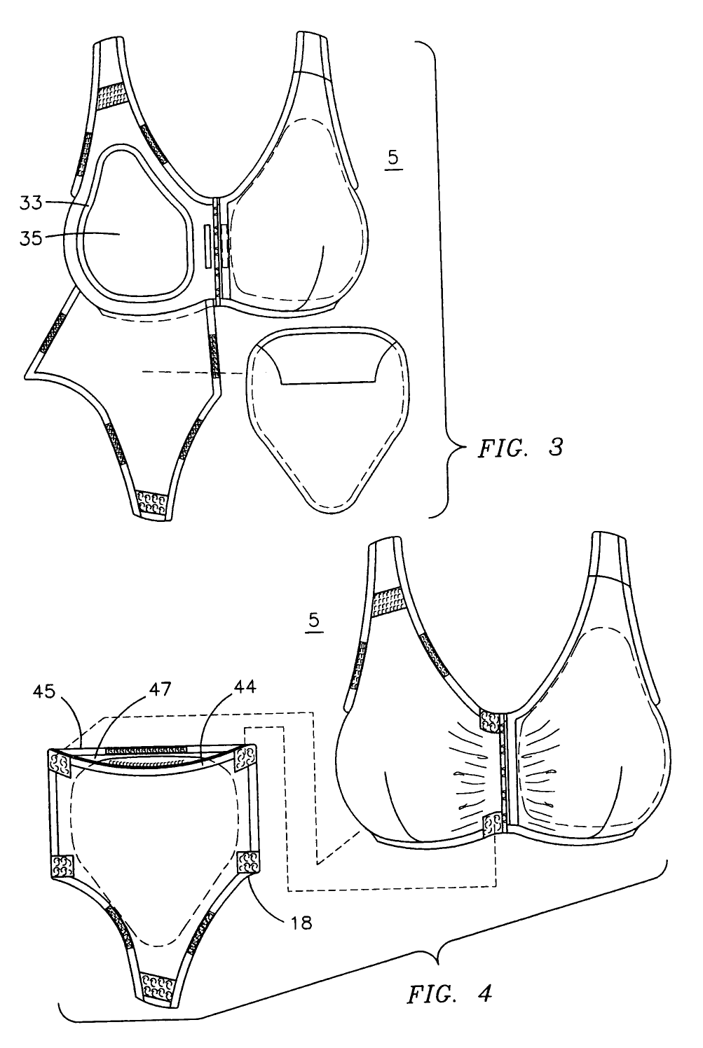

A flap of material 16 covers the left bra cup 14 of the bra 5 and a second flap of material 18 covers the right bra cup 12 of the bra 5. The flaps 16, 18 are slightly larger than the bra cups 12, 14, but comprise the same shape as the bra cup 12, 14. One skilled in the art will also recognize that the flaps 16, 18 may also comprise elastic to create a shape to better conform to the respective bra cups 12, 14. Due to discomfort that may be realized by movement of a breast, the flaps 16, 18 can be opened and closed with little to no movement of the breast, thus allowing the changing of gel packs 20 with minimum movement and discomfort to the patient. In a preferred embodiment, as illustrated in FIG. 1, the flaps of material 16, 18 are permanently connected to the bra 5 along an outer edge 22 of each respective bra cup 12, 14, such as along a lower edge 22 of the bra 5. Connection attachments 25 are provide to secure each flap around a respective breast cup 12, 14. In a preferred embodiment, the connection attachments 25 are Velcro™. In another preferred embodiment, as disclosed in FIG. 3, the flaps 16, 18 are not permanently connected, but are removable from the bra 5. As illustrated, a single closure device 27 is provided to secure a top end 28 of the flaps 16, 18 to a strap 30 of the bra 5. To better support a therapeutic insert 20, the outer edge of the flaps 16, 18 comprises a plurality of closure devices 25, displaced around the flaps 16, 18 and the outer edge of the bra cups 12, 14.

A pouch area is formed between each respective bra cup 12, 14 and flaps 16, 18. A therapeutic insert 20, or gel pack, is positioned within the pouch area. The gel pack 20 has a pre-formed, but pliable, shape to form to a shape of a breast. In a preferred embodiment, depending on a breast size, a plurality of preformed gel packs 20 are provided wherein a user can select an appropriate size that best fits a user. The gel pack 20 is made of a material that, when placed in a cold environment such as a freezer, will retain the cold temperature for an extended length of time. Likewise, when placed in a warm or heated environment, such as heating by a microwave, the gel pack 20 will retain its heated state for an extended period of time.

As further illustrated in FIG. 2, the bra cup 12, 14, or front panel directly covering a breast, can be opened to expose the breast to air. In another preferred embodiment, illustrated in FIG. 3, an opening 32 is provided in the front panel 12, 14 exposing the breast to air when the flap 16, 18 and insert 20 are not in place. The opening 32 may consist of a plurality of shapes. The opening 32 is further enforced with stitching 33 around the opening 32, or another reinforcing device, such as additional material, so that the breast is held in position when exposed.

As further illustrated in FIGS. 1-4, in a preferred embodiment, the gel pack 20 is fitted within a sleeve 35. The sleeve 35 is provided to absorb any condensation or liquid that may be produced from the gel pack 20, so as to keep the wearer from experiencing any discomfort as a result of liquid making contact with the body or the bra 5 directly. In a preferred embodiment, the sleeve 35 is made of soft, breathable material, with a water-repellant interior.

A back panel, or strip, not shown, is also provided which has a first end connected to the first front panel 12 and a second end connected to the second front panel 14. Though disclosed as a front closure bra, in another preferred embodiment, not shown, the therapeutic bra 5 is a back closure bra. In this embodiment, the closure device is part of the back panel.

FIG. 4 also discloses another way that the gel packs 20 may be used with the present invention 5. As disclosed, the gel packs 20 are not enclosed in a sleeve. Instead the flaps 12, 14 are made of two pieces of material 44, 45 and have an opening 47 between the adjacent materials 44, 45 where the gel packs 20 are placed. Though not illustrated the sleeve 35 could still be used if desired.

In a preferred embodiment, the therapeutic bra 5 is made of two-way, breathable, stretch material. The front closure device 10 is a standard hook-and-eye closure system. The bottom band 22 of the bra 5, where the flaps 16, 18 are connected in a preferred embodiment, has an elastic band to ensure proper support without the use of an under wire, since the use of an under wire is not recommended by plastic surgeons. The shoulder straps 30, 31 are elastic or adjustable so they can be adjusted to accommodate various users or wearers. The flaps 16, 18 are made of two-way, breathable, stretch material, and are slightly larger than the bra cups 12, 14. The connection 40, as illustrated in FIG. 1, along the bottom of the bra 5 may be from the center 42 of the bra 5, where the bra 5 opens and closes, to just before the arm openings 44.

The gel packs 20 comprise a non-freezing, non-toxic, colorless, pliable gel material. The gel packs 20 are configured so that the material inside is evenly disbursed within the gel pack. As previously discussed, the gel packs 20 are shaped to the form to a breast. Having this shape will facilitate even cooling and/or warming (or heating) of an entire breast area.

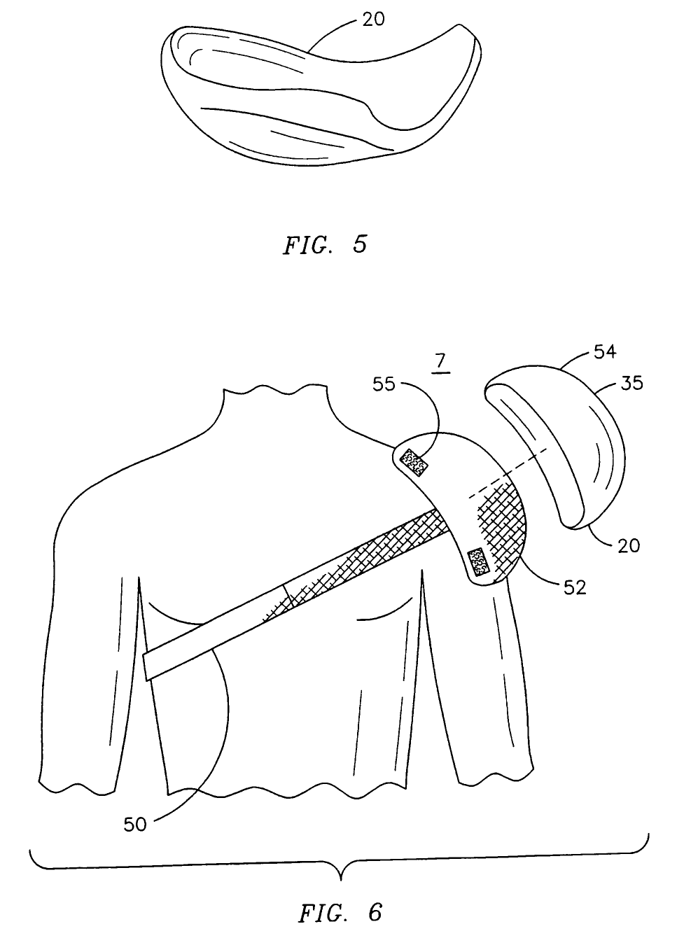

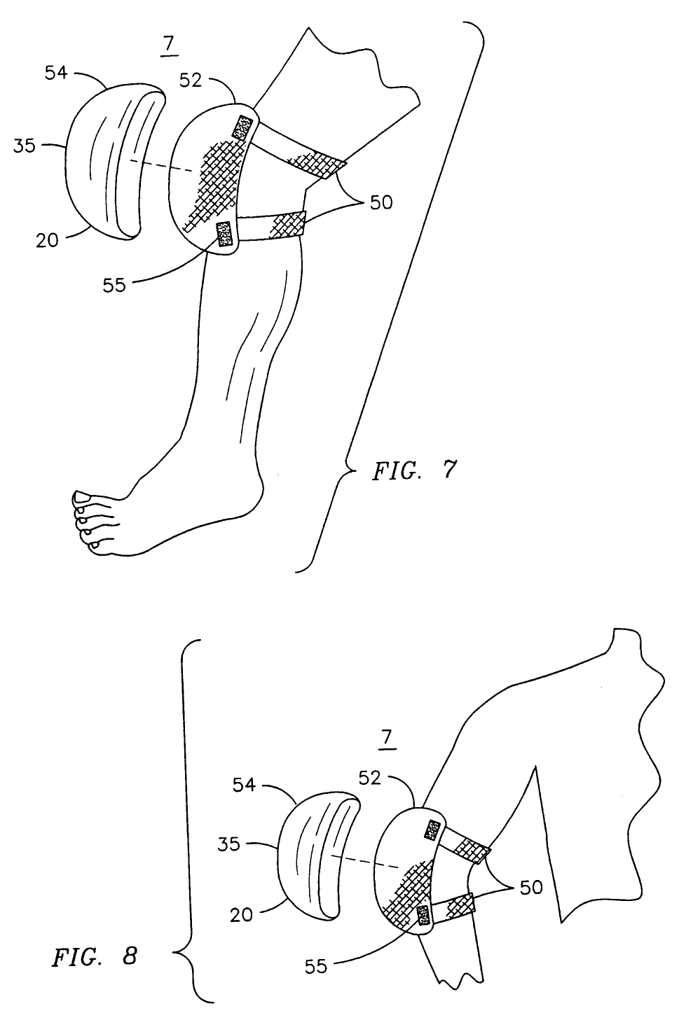

FIGS. 6, 7, and 8 are exemplary embodiments of the therapeutic body covering 7 being applied to other areas of a body, such as a shoulder, knee, and elbow respectively. As illustrated a strap 50, or a plurality of straps 50, are provided to secure a panel 52 to either the shoulder, knee or elbow. Though not illustrated, the present invention can also be used with an ankle. These straps 50 are adjustable to insure that the panel 52 fits properly to the body area desired. The adjustability of these straps 50, or securing straps, can include elastic material and/or a clasp or Velcro™ connectors. In another embodiment, the adjustable straps are made of a cotton base material.

A removable panel 54 is also provided. In one embodiment, the removable panel 54 is the therapeutic sleeve 35, discussed above, which has connection points 55, such as Velcro™ to allow it to be attached and removed from the panel 52. In another embodiment, the gel pack 20, including the therapeutic sleeve 35, if that option is desired, fits between the panel 52 and the removable panel 54. The shape of the gel pack 20 has a convex, or bowl shape, that is oval in length. As discussed above, the gel pack 20 is available in a plurality of sizes so that a specific size provides a best fit to a body region.

While the preferred embodiments of the present invention have been shown and described herein, it will be obvious that such embodiments are provided by way of examples only. Numerous variations, changes and substitutions will occur to those of skill in the art without departing from the invention herein. Accordingly, it is intended that the invention be limited only by the spirit and scope of the appended claims.

What we claim is:

1. A therapeutic body covering specific to a certain area of a body, said body covering comprising:
(a) a panel for covering a certain area of a body;
(b) a removable flap positioned over said panel;
(c) a therapeutic insert configured to conform to said area of said body;
(d) a securing panel to hold said panel to said certain area of said body; and
(e) wherein said panel and said securing panel are configured so that said certain area of said body is not moved when placing said therapeutic insert between and removing said therapeutic insert from said panel and said flap.

2. The therapeutic body covering of claim 1 wherein said removable panel further comprises a second removable panel to form a therapeutic sleeve that has an opening to receive said therapeutic insert.

3. The therapeutic body covering of claim 2 wherein said therapeutic sleeve further comprises a liquid repellant inner liner.

4. The therapeutic body covering of claim 1 wherein said therapeutic insert comprises a material that maintains at least one of a cooling temperature for an extended period of time and a warming temperature for an extended period of time.

5. The therapeutic body covering of claim 1 wherein said therapeutic insert comprises a preformed shape that conforms to a shape of said certain area of said body.

6. The therapeutic body covering of claim 1 wherein said securing panel is adjustable to allow said therapeutic body covering to best fit to said certain area of said body.

7. The therapeutic body covering of claim 1 wherein said certain area of said body comprises at least one of a knee area, a shoulder area, an elbow area, an ankle area, and a mammary gland area.

8. A therapeutic bra, said bra comprising:
(a) a first front panel for covering a first breast;
(b) a second front panel for covering a second breast;
(c) a closure device holding said first panel and said second panel together;
(d) a back panel having a first end connected to said front panel and a second end connected to said second panel;
(e) a removable first flap positioned over said first front panel;
(f) a removable second flap positioned over said second front panel;
(g) a therapeutic insert;
(h) wherein said therapeutic insert is configured to conform to a breast size; and
(i) wherein said first front panel, said second front panel, and said back panel are configured so that said breasts are not moved when placing said therapeutic insert between and removing said therapeutic insert from at least one of said first front panel with said first flap and said second front panel with said second flap.

9. The therapeutic bra of claim 8 further comprises a therapeutic sleeve fitted around said therapeutic insert.

10. The therapeutic bra of claim 9 wherein said therapeutic sleeve further comprises a liquid repellant inner liner.

11. The therapeutic bra of claim 8 wherein each of said flaps is permanently affixed to each of said respective panels wherein said first flap opens to allow at least one of a placement and a removal of said therapeutic insert between said first flap and said first panel without moving said breast and said second flap opens to allow at least one of placement and removal of said therapeutic insert between said second flap and said second panel without moving said breast.

12. The therapeutic bra of claim 8 further comprises a first strap and a second strap wherein said first strap has a first end connected to said first panel and a second end connected to said back panel and said second strap has a first end connected to said second panel and a second end connected to said back panel.

13. The therapeutic bra of claim 8 wherein detachable fasteners connect said first flap to said first panel and said second flap to said second panel.

14. The therapeutic bra of claim 8 wherein at least one of said first panel and said second panel further comprise an opening to allow for access to a breast nipple when at least one of said first flap and said second flap are removed.

15. The therapeutic bra of claim 8 wherein at least one of said first panel and said second panel comprises two pieces of material wherein an opening is provided between said pieces of material for placement of said therapeutic insert.

16. The therapeutic bra of claim 15 wherein said opening further comprises a reinforced edge around said opening to further prevent movement of said breast when at least one of removing said therapeutic insert and inserting said therapeutic insert.

17. A therapeutic bra for reducing movement of a breast when applying at least one of a cooling compress and a warming compress to said breast, said therapeutic bra comprising:
(a) a first bra cup covering a first breast;
(b) a second bra cup covering a second breast;
(c) a first bra cup flap removable from an outer edge of said first bra cup;
(d) a second bra cup flap removable from an outer edge of said second bra cup;
(e) a back strap having a first end connected to said first bra cup and a second end connected to said second bra cup;
(f) at least one of a cooling compress and a warming compress formed to a shape of at least one of said first breast and said second breast; and
(g) wherein at least one of said cooling compress and said warming compress fits between at least one of said first bra cup flap connected to said first bra cup and said second bra cup flap connected to said second bra cup.

18. The therapeutic bra of claim 17 wherein at least one of said cooling compress and said warming compress are formed to contour to at least one of said breasts.

19. The therapeutic bra of claim 17 wherein said first bra cup flap has a first edge that is permanently affixed to a first edge of said first bra cup and wherein said second bra cup flap has a first edge that is permanently affixed to a first edge of said second bra cup.

20. The therapeutic bra of claim 17 wherein at least one of said first bra cup and said second bra cup further comprises an opening in said bra cup to allow exposure of said breast.

21. The therapeutic bra of claim 17 further comprises fasteners to connect at least one of said first bra cup flap to said first bra cup and said second bra cup flap to said second bra cup.

22. The therapeutic bra of claim 17 further comprises a front closure device connecting said first bra cup to said second bra cup along adjacent edges of each respective bra cup.

23. The therapeutic bra of claim 17 further comprises a first strap having one end connected to said first bra cup and a second end connected to said back panel and a second strap having one end connected to said second bra cup and a second end connected to said back panel.

24. The therapeutic bra of claim 23 wherein said straps are adjustable.

25. The therapeutic bra of claim 17 further comprises a sleeve that fits around at least one of said cooling compress and said warming compress.

26. The therapeutic bra of claim 17 wherein said cooling compress and said warming compress are a single compress having a material that can be both cooled to maintain a given cool temperature for an extended period of time and warmed to maintain a given warm temperature for an extended period of time.

27. The therapeutic bra of claim 17 wherein at least one of said first flap and said second flap comprises two pieces of material wherein an opening is provided between said pieces of material for placement of said therapeutic insert.

28. An improvement to a bra having a first bra cup and a second bra cup, said improvement comprising:
(a) a first movable flap covering a first bra cup;
(b) a second movable flap covering a second bra cup;
(c) therapeutic inserts that fits between each first flap in combination with said first bra cup and said second flap in combination with said second bra cup; and
(d) wherein each said flap can be opened for insertion and removal of said therapeutics where a breast is not moved.

29. The improvement of claim 28 further comprising said therapeutic insert being conformed to a shape of a breast.
